(19) United States
(12) Patent Application Publication
(10) Pub. No.: US 2025/0064477 A1
(43) Pub. Date: Feb. 27, 2025

(54) SYSTEMS AND METHODS FOR IRRIGATING AN ANATOMIC SPACE

(71) Applicant: The General Hospital Corporation, Boston, MA (US)

(72) Inventors: Jonathan Kusner, Boston, MA (US); Graham Lieberman, Boston, MA (US); Avik Som, Boston, MA (US); Brian Eisner, Needham, MA (US); Shouvik Das, Boston, MA (US); Gokul Prasath Rajamanickam, Cambridge, MA (US); John Di Capua, Boston, MA (US)

(73) Assignee: The General Hospital Corporation, Boston, MA (US)

(21) Appl. No.: 18/726,745

(22) PCT Filed: Jan. 3, 2023
(86) PCT No.: PCT/US2023/060010

§ 371 Date: Jul. 3, 2024

Related U.S. Application Data
(60) Provisional application No. 63/296,091, filed on Jan. 3, 2022.

(51) Int. Cl. A61B 17/3203; A61B 17/22; A61B 17/32; A61B 90/00

(52) U.S. Cl. CPC A61B 17/32037; A61B 2017/22065; A61B 2017/22072; A61B 2017/22079; A61B 2017/320024; A61B 2090/036; A61B 2090/064; A61B 2217/007

(57) ABSTRACT

Systems and methods are provided that may facilitate the irrigation of an anatomic space. The irrigation system can include a tube manifold, a fluid reservoir, a waste fluid trap and an emulsification system. The emulsification system can be arranged at a distal end portion of the tube manifold and can be configured to be inserted into an anatomic space and emulsify organic material located within the anatomic space.

### CROSS-REFERENCES TO RELATED APPLICATIONS

This application claims priority to U.S. Provisional Application No. 63/296,091 filed on Jan. 3, 2022, which is hereby incorporated by reference herein in its entirety.

### STATEMENT REGARDING FEDERALLY SPONSORED RESEARCH

Not Applicable.

### BACKGROUND OF THE INVENTION

Gross hematuria is a condition in which visible blood is present in the urine. It can be caused by bleeding from urologic malignancy or friable vessels within a hypertrophic prostate. When the level of blood builds up to a high enough concentration, it forms clot(s) within the bladder that can obstruct the outflow of urine. When the outflow of urine from the bladder is blocked, the bladder continues to fill with urine with inability by the patient to void. This is a medical urgency/emergency that often requires acute decompression of the bladder as well as manual irrigation to remove as much clot burden from the bladder as possible to ensure a patent and non-obstructed urinary outflow tract.

### SUMMARY OF THE INVENTION

In some aspects, the present disclosure provides systems and methods that may facilitate the irrigation of an anatomic space. The irrigation system can include a tube manifold, a fluid reservoir, a waste fluid trap, and an emulsification system. The emulsification system can be arranged at a distal end portion of the tube manifold and can be configured to be inserted into an anatomic space and emulsify solid and semi-solid organic material located within the anatomic space.

In another aspect, the present disclosure provides a method of hematuria irrigation. The method can include inserting a catheter into a bladder of a patient and introducing an irrigant into the bladder provided by a fluid source and propelled by a fluid module. The method can include controlling an emulsifier to engage solid and semi-solid organic material within the bladder. The method can include draining the irrigant and organic material from the bladder into a waste fluid trap via a vacuum module.

In another aspect, the present disclosure provides a method of operating an irrigation system. The method can include, in a nominal mode, flushing an anatomic space with a fluid and creating a flow having a shear force to break down and evacuate a clot or other organic material. In a maintenance mode, the method can include continuing to flush the anatomic space and creating a flow that is turbulent, laminar, or a combination thereof to prevent a clot from re-forming or other organic material from accumulating. In a troubleshooting mode, the method can include alternating between the nominal mode and the maintenance mode to clear out catheter clogs.

In some aspects, the present disclosure provides an irrigation system having a fluid recycler. The irrigation system can include a pump, a pressure regulator, a fluid reservoir configured to receive patient aspirate and irrigant, a filter in fluid communication with the fluid reservoir to separate the patient aspirate from the irrigate, a port fluidly coupled to a suction generator, and a pressure sensor configured to monitor pressure of the recycler system.

In some aspects, the present disclosure provides a handheld device for use with a fluid recycler system. The handheld device can include a first channel fluidly coupled to a fluid reservoir of the recycler system in an inflow passageway, a second channel fluidly coupled to the fluid reservoir in an outflow passageway, a fluid chamber in fluid communication with each of the first channel, the second channel, and catheter, and a control switch. The control switch can be configured to selectively allow for fluid flow through the inflow passageway via the first channel and can allow for fluid flow through the outflow passageway via the second channel.

The foregoing and other aspects and advantages of the disclosure will appear from the following description. In the description, reference is made to the accompanying drawings which form a part hereof, and in which there is shown by way of illustration a preferred configuration of the disclosure. Such configuration does not necessarily represent the full scope of the disclosure, however, and reference is made therefore to the claims and herein for interpreting the scope of the disclosure.

### BRIEF DESCRIPTION OF THE DRAWINGS

The invention will be better understood and features, aspects and advantages other than those set forth above will become apparent when consideration is given to the following detailed description thereof. Such detailed description makes reference to the following drawings.

FIG. 1 is a schematic illustration of an irrigation system in accordance with the present disclosure.

FIG. 2 is a schematic illustration of a catheter end of the irrigation system of FIG. 1.

FIG. 3A is a schematic illustration of another catheter end in accordance with the present disclosure.

FIG. 3B is a schematic illustration of the catheter end of FIG. 3A including an agitator.

FIG. 3C is a schematic illustration of the catheter end of FIG. 3B with the agitator in a deployed position.

FIG. 4 is a schematic illustration of another irrigation system in accordance with the present disclosure.

FIG. 5 is a flowchart illustrating a method of using an irrigation system.

FIG. 6 is a schematic illustration of a fluid recycler having a canister filter in accordance with the present disclosure.

FIG. 7 is a schematic illustration of the fluid recycler of FIG. 6 having an in-line filter.

FIG. 8 is a schematic illustration of the fluid recycler of FIG. 6 having a filter pump.

### DETAILED DESCRIPTION OF THE INVENTION

The following discussion is presented to enable a person skilled in the art to make and use aspects of the present disclosure. Various modifications to the illustrated configurations will be readily apparent to those skilled in the art, and the generic principles herein can be applied to other configurations and applications without departing from aspects of the present disclosure. Thus, aspects of the present disclosure are not intended to be limited to configurations shown, but are to be accorded the widest scope consistent with the principles and features disclosed herein. The following detailed description is to be read with reference to the figures, in which like elements in different figures have like reference numerals. The figures, which are not necessarily to scale, depict selected configurations and are not intended to limit the scope of the present disclosure. Skilled artisans will recognize the non-limiting examples provided herein have many useful alternatives and fall within the scope of the present disclosure.

As described above, when blood clots form within a bladder, the outflow of urine may be obstructed. In some cases, irrigation is required to remove the obstruction. Manual irrigation is typically performed by emergency physicians and urologists. There are variations in how irrigation can be performed, but generally a manual irrigation includes urinary catheterization followed by repeated instillation and aspiration of sterile water/saline through the catheter with a large volume (50-60cc) catheter-tip syringe. It is estimated that approximately 750,000-1 million hematuria irrigations are required in the United States on a yearly basis.

The process generally evacuates small pieces of clot from the bladder through the catheter over and over again. Care must be taken during the process to apply enough force to break down and evacuate the clot without suctioning the tissue of the bladder, prostate, or urethra. Care must also be taken to keep the bladder partially full, such that the catheter can suction the clot without suctioning tissue, but not so full that the patient is uncomfortable or at risk of bladder overdistention. In some instances, an irrigation process may be repeated until the evacuated fluid is clear, free of clot, and is not diluted with blood, indicating the clot has been fully evacuated. Such processes can take several hours.

Patients who have undergone the irrigation process often stay overnight in a hospital, and in some cases, require additional or continuous bladder irrigation to clear any additional bleeding from the bladder and help break down any residual clots. In some cases, a patient may be unable to be cleared of clots via manual irrigation alone and will continue to have clot pieces obstruct their catheter, requiring a cystoscopy in the operating room for cystoscopic clot evacuation and/or cautery of bleeding sources.

Aspects of the present disclosure overcome these and other drawbacks of an irrigation procedure. For example, embodiments of the present disclosure automates portions of an irrigation process which can decrease the time required to perform a proper irrigation, decrease the complexity of hematuria irrigations, and increase the number of patients who are properly irrigated the first time such that they require less repeated manual irrigation or cystoscopic clot evacuation. Aspects of the present disclosure may allow a broader range of medical professionals to perform an irrigation and decrease the number of days a patient spends in a hospital.

Although embodiments of the disclosure described below are in reference to hematuria irrigation, it should be understood that embodiments described herein may be used in additional processes. For example, embodiments of the present disclosure may be used in non-operatively removing devitalized prostate tissue from the bladder after a prostate enucleation or transurethral resection of prostate to save operating room and surgeon/anesthesiologist time; irrigating necrotic pancreatic pseudo-cysts in chronic pancreatitis requiring irrigation drains (that often clog) typically placed by interventional radiology or general surgery specialists; irrigating gastrostomy, jejunostomy, and other percutaneous gastrointestinal tubes at risk of clogging with foodstuff or intestinal contents; irrigating and draining abscesses; and other clinical or surgical scenarios in which organic materials must be evacuated from an anatomic space.

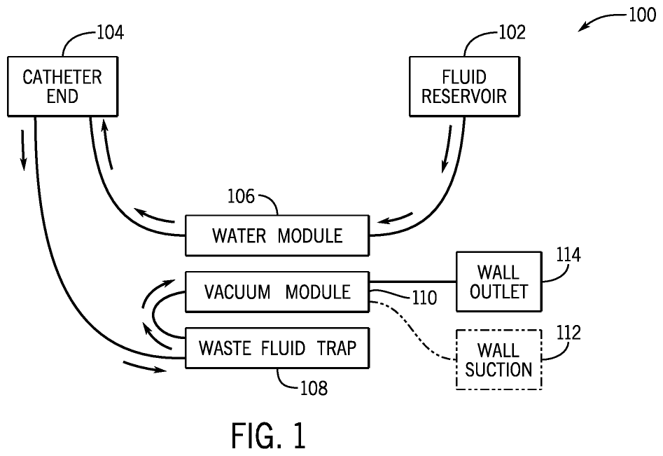

FIG. 1 illustrates an irrigation system 100 according to some embodiments of the disclosure. The irrigation system 100 includes a fluid reservoir 102 in fluid communication with a catheter having a catheter end 104. The fluid reservoir 102 and the catheter end 104 are in fluid communication with a water module 106. In some embodiments, the water module 106 is configured as a flow module. The flow module can be motorized and may be configured as a pump to provide fluid flow from the fluid reservoir 102 to the catheter end 104.

The irrigation system 100 includes a waste fluid trap 108 in fluid communication with a vacuum module 110. In some embodiments, one of the waste fluid trap 108 or the vacuum module 110 can be in fluid communication with a wall suction system 112 often found in hospitals. Additionally, the irrigation system 100 can include a power source. In the illustrated embodiment, the power source is configured as a wall outlet 114. However, other power sources are possible, such as a battery, for example.

In use, the fluid reservoir 102 is hung, or otherwise disposed, above a patient. The fluid reservoir 102 can include a bag of saline, such as a 3L saline irrigation bag, for example. In general, fluid from the fluid reservoir 102 can travel through tubing and into the water module 106. The water module 106 can send fluid to the catheter end 104 which may be inserted into a bladder of the patient. The water module 106 can provide a flow, (e.g., a pulsatile flow) to the bladder. The catheter end 104, which is seated inside the bladder, allows inflow and drainage of fluid.

The fluid that flows from the fluid reservoir 102 can provide a combination of turbulent flow, laminar flow, and shear force to break down clots proximate to the catheter end 104 within the bladder. The flow of fluid can emulsify one or more blood clots within the bladder. A negative pressure can be provided in the waste fluid trap 108 to remove the emulsified clot, which can be generated by the vacuum module 110 or the wall suction 112, depending on the particular setup and availability of a hospital room.

In some embodiments, the catheter end 104, the vacuum module 110, and the water module 106 can each include sensors. Such sensors can sense one or more fluid parameters, such as pressure and flowrate, for example. The sensors can cause the irrigation system 100 to variably switch between operating modes. For example, operating modes of the irrigation system 100 can include a nominal mode, a maintenance mode, and a troubleshooting mode. In the nominal mode, the irrigation system 100 works to break down and evacuate a clot from the bladder. In the maintenance mode, the irrigation system 100 can provide alternating positive and negative pressure to irrigate and flush the bladder to prevent a clot from re-forming. In the troubleshooting mode, the irrigation system 100 may be switched between the nominal mode and the maintenance mode to clear out catheter clogs.

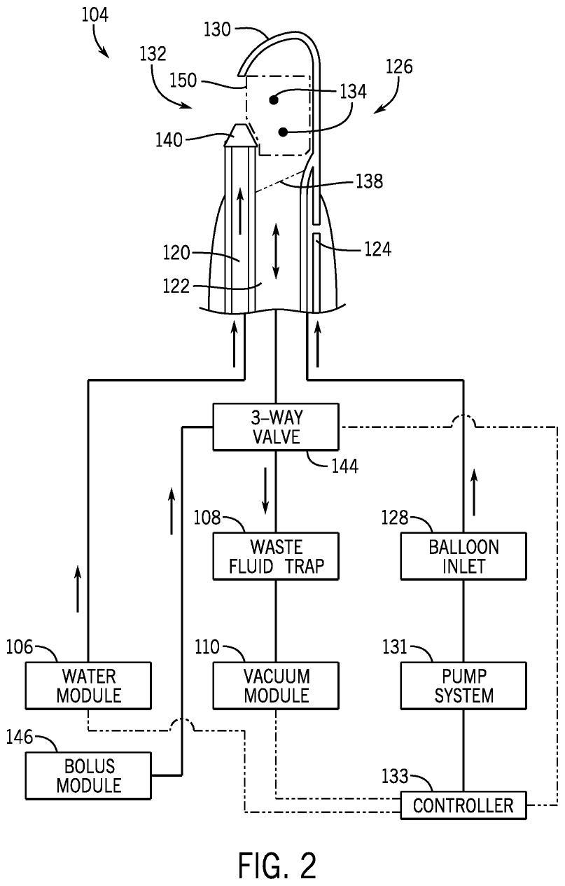

FIG. 2 illustrates one non-limiting example of the catheter end 104. In the illustrated embodiment, the catheter is configured as a triple-lumen catheter. The catheter end 104 includes a first channel 120, a second channel 122, and a third channel 124. The first channel 120 can receive an inflow of fluid and is in fluid communication with the water module 106. The second channel 122 is in fluid communication with the waste fluid trap 108 and the vacuum module 110. The waste fluid trap 108 can collect emulsified clots and a negative pressure can be maintained by the vacuum module 110.

As briefly described above, in use, a tip 126 of the catheter end 104 is seated within the bladder of the patient. The third channel 124 provides a balloon inlet 128 so that a catheter balloon 130 can be inflated within the bladder and wrap circumferentially around the catheter tip 126 to help ensure that the catheter end 104 remains indwelling within the bladder. In some embodiments, the catheter balloon 130 can expand farther in a radial direction than in an axial direction so that the catheter balloon 130, when inflated, forms a discoid. The catheter balloon 130 can be inflated with a fluid, such as air. When the catheter balloon 130 is deflated, it can be flat against the catheter. In some embodiments, the catheter balloon 130 can be used to sense pressure within the anatomic space. One or more pressure parameters can be selected and used to dictate the positive or negative pressure applied to the anatomic space. For example, a pump system 131 may be connected to the balloon inlet 128 to inflate the catheter balloon 130.

The pump system 131 may be programmable, or be controlled by a processor or controller system 133, whether dedicated to the pump system 131 or coordinating operation of other systems, such as the vacuum module 110 or other components of the whole system 100. Regardless of the particular hardware and software control system, the pump system 131 may be programmed or controlled to sense a pressure applied to the catheter balloon 130 while deployed and use such pressure sensing as feedback for the control of the catheter balloon 130, but also other components of the system 100. For example, the pump system 131 or controller 133 can be used to determine that the 3-way valve 144 or the water module 106 should be adjusted, such as to reduce pressure in the anatomic space.

The catheter end 104 can include an emulsification system 132. In general, the emulsification system 132 is configured to break up or otherwise distribute portions of organic material, such as clots, to facilitate draining the organic material from an anatomic space (e.g., the bladder). In one non-limiting example, the emulsification system 132 can include cutting or shredding elements 134, which may include rigid bars, wires, or blades (shown in cross-section in FIG. 2) that act as static turbulent flow generators which can enhance clot breakdown as fluid flows through the second channel 122 toward the waste fluid trap 108. For example, as fluid flows past the cutting or shredding elements 134, the flow can transition to a turbulent flow to help break up clots. The cutting or shredding elements 134 can additionally or alternatively be used to help break up clots. As fluid flows past the cutting or shredding elements 134, clots within the fluid can engage the cutting or shredding elements 134 and become emulsified in the fluid and drained to the waste fluid trap 108. In other embodiments, the emulsification system 132 can include a network of one or more thin wires or mesh that is held firmly in place in the catheter end 104 as fluid flows through the catheter end 104.

Also, the emulsification system 132 can additionally or alternatively include a cutting or shredding element 138 or other component configured to cut, shred, or otherwise reduce the size of a material. In the illustrated embodiment, the cutting element 138 may be a blade that can be positioned within the second channel 122 and configured to break up solid or semi-solid organic material that flows through the second channel 122, for example, to help prevent clogs from forming. Elements of the emulsification system 132, including the rigid bars 134 and/or the blade element 138, can be constructed from a variety of materials, such as nylon, metals, natural fibers, etc.

Still referring to FIG. 2, some embodiments of the emulsification system 132 can include a nozzle 140 that provides a narrowed channel at the distal end of the first channel 120. The nozzle 140 can be configured to accelerate fluid flow from a fluid source, such as the water module 106. The nozzle 140 can provide an emulsifying jet of irrigant to an anatomic space to break down unwanted solid and semi-solid material.

Thus, the emulsification system 132 can include a combination of components, such as cutting or shredding elements 134, 138, and nozzle 140 arranged as described in FIG. 2 or in other parts of the system 100. For example, mesh wires, bars, a blade, or a nozzle may be used together or alone. Additionally, one or more elements of these or other elements of the emulsification system 132 can be configured to vibrate to further facilitate the breakdown of solid and semi-solid organic material.

Some embodiments of an irrigation system 100 can further include a three-way valve 144. As illustrated in FIG. 2, the three-way valve can be in fluid communication with the second channel 122 of the catheter end 104, a vacuum trap or waste fluid trap 108, and a bolus module 146. In such embodiment, the three-way valve 144 can be actuated between a first position and a second position. In the first position, the second channel 122 is in fluid communication with the vacuum trap or waste fluid trap 108, and thereby the vacuum module 110. In the second position, the second channel 122 is in fluid communication with the bolus module 146. In some embodiments, the bolus module 146 can provide a secondary fluid source that is separate from the water module 106 and can provide a positive pressure within the tube manifold.

Further, some embodiments of the catheter end 104 can include a reinforcement structure 150. The reinforcement structure 150 can be positioned proximate to an outlet of the catheter end 104 to add rigidity to the catheter end 104. In some embodiments, the reinforcement structure 150 can be integrally formed with the catheter end 104, or may be formed as a sheath that surrounds the catheter end 104. In general, the reinforcement structure is configured to withstand the force produced by the vacuum module 110 proximate to the catheter end 104 and prevent any of the first, second, and third channels 120, 122, 124 from collapsing or impeding access to the lumen of the catheter.

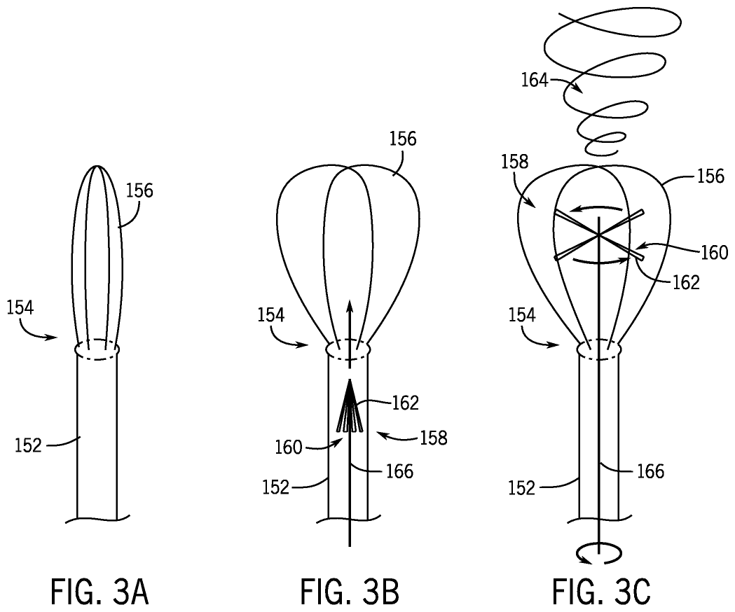

FIGS. 3A-3C illustrate a non-limiting example of a catheter 152 having a catheter end 154. In some embodiments, the catheter end 104 of the irrigation system 100 can include some or all of the features of the catheter end 154. Like the catheter end 104, the catheter end 154 can include an emulsification system 158. In one non-limiting example, as illustrated in FIG. 3A, the emulsification system 158 can include a cage 156. As illustrated in FIG. 3B, the cage 156 can be expandable. In some embodiments, the cage 156 can act as an emulsifier during an irrigation and drainage process by engaging and breaking up unwanted organic material.

In another non-limiting example, as illustrated in FIGS. 3B and 3C, the emulsification system 158 can also include a mechanical agitation system 160. In the illustrated example, when the catheter end 154 is seated in the bladder of a patient, the cage 156 can exit the end of the catheter 152 and expand within the bladder. The mechanical agitation system 160 can be deployed through the catheter 152, as illustrated in FIG. 3B, and into the bladder. In the illustrated embodiment, the mechanical agitation system 160 is configured as a rotary agitator and includes collapsible blades 162 that can expand within the cage 156.

As illustrated in FIG. 3C, the blades 162 of the mechanical agitation system 160 can expand within the cage 156 and spin to generate fluid movement within the bladder. In the illustrated embodiment, the mechanical agitation system 160 can create a turbulent flow 164 or other shear forces within the bladder. The blades 162 of the mechanical agitation system 160 are disposed at an end of a semi-rigid wire 166 that is configured to rotate the blades 162 rapidly when actuated. In other embodiments, the mechanical agitation system 160 can include additional or alternative components to agitate fluid, such as a rotor, fan, or flap, for example.

As described above, the cage 156 can act as an emulsifier and additionally or alternatively prevent the blades 162 of the mechanical agitation system 160 from impacting boundaries in an anatomic space. In particular, the cage 156 allows free-floating organic material, such as a blood clot within the bladder, to come in close proximity to the catheter end 154 while the mechanical agitation system 160 is prohibited by the cage 156 from engaging any fixed tissue boundaries or walls of the bladder.

In general, the mechanical agitation system 160 is disposed near the catheter end 154 and can morcellate clots either within the catheter 152 or beyond the catheter end 154 within the cage 156. The emulsification system 158 can expedite an irrigation process by emulsifying clots while being unable to mechanically damage a bladder, prostate, or urethra when inserted into a patient. In some embodiments, the mechanical agitation system 160 may be powered via a power source, such as the wall outlet 114 in the irrigation system 100. In other embodiments, the mechanical agitation system 160 can be powered pneumatically, via a suction source such as a vacuum pump, vacuum module 110, or wall suction 112 described above, or via a battery.

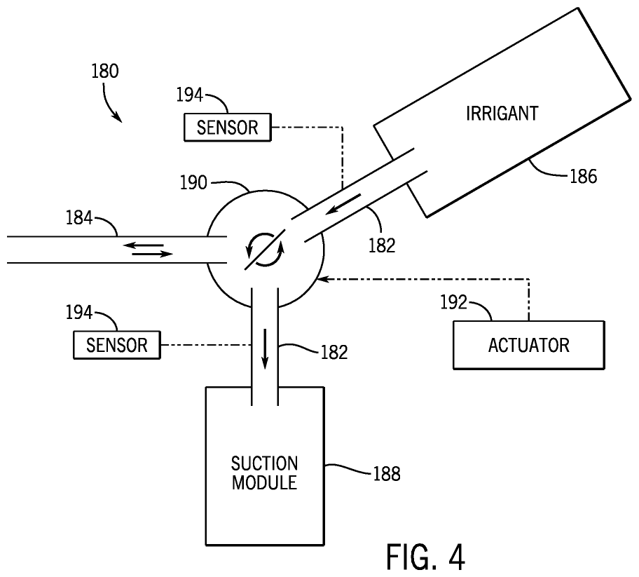

FIG. 4 illustrates an irrigation system 180 according to another embodiment of the disclosure. The irrigation system 180 includes a tube manifold 182 which includes a catheter 184. The catheter 184 can include an end that is received within a bladder of a patient, such as the catheter end 104 or the catheter end 154 described above. The irrigation system 180 includes a fluid or irrigant reservoir 186, a vacuum or suction module 188, and a valve 190. In the illustrated embodiment, the valve 190 is configured as a three-way valve in fluid communication with the catheter 184 and selectively in fluid communication with the irrigant reservoir 186 and the suction module 188.

The valve 190 may be actuated via an actuator 192. The actuator 192 can switch the valve between an open position relative to the irrigant reservoir 186 and an open position relative to the suction module 188. In particular, the actuator 192 can actuate the valve 190 to cause a switch between a positive pressure and a negative pressure (or vice versa) within the tube manifold 182. In use, an operator may insert the catheter into a bladder of a patient and irrigate the bladder by sending irrigant, such as a saline solution, through the tube manifold 182 and the catheter 184. The operator may then use the actuator 192 to switch the valve 190 so that the suction module 188 can drain fluid, and possibly clots or other organic material, from the bladder or anatomic space. The operator may switch the valve 190 via the actuator 192 a plurality of times during an irrigation process to flush and drain fluid through a bladder. In some examples, the actuation may be rapid.

In some non-limiting examples, the actuation may be automated. For example, the irrigation system 180 can include one or more sensors 194 in communication with the tube manifold 182 or catheter 184. The sensors 194 can sense fluid parameters such as pressure and flowrate, for example. The sensors 194 may be able to sense fluid flowing to or from the bladder and can indicate to the operator to actuate the valve 190. In another example, the sensors 194 can automatically provide feedback to and control the actuator 192 and the sensors 194 can signal to the actuator 192 to actuate the valve 190 in response to a predetermined threshold.

In some implementations, devices or systems disclosed herein can be utilized using methods embodying aspects of the disclosure. Correspondingly, description herein of particular features, capabilities, or intended purposes of a device or system is generally intended to inherently include disclosure of a method of using such features for the intended purposes and a method of implementing such capabilities. Unless otherwise indicated or limited, discussion herein of any method of using a particular device or system, including installing the device or system, is intended to inherently include disclosure, as embodiments of the invention, of the utilized features and implemented capabilities of such device or system.

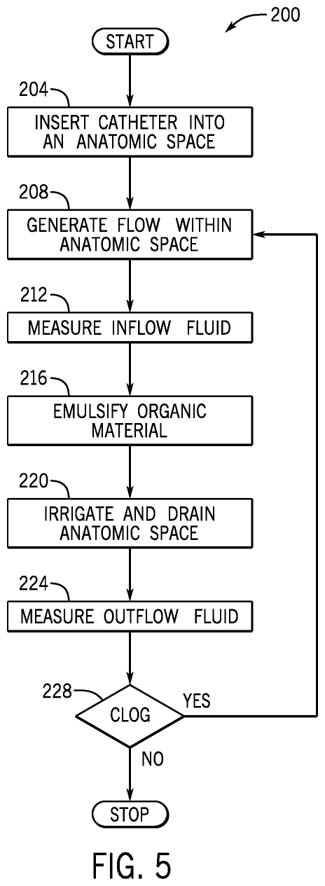

In this regard, for example, FIG. 5 illustrates a method 200 for performing irrigation within an anatomic space, such as a bladder, for example. By way of example, the method 200 will be described below with reference to the irrigation system 100, although other systems, including the irrigation system 180, can be used. Operation 204 of method 200 includes inserting the catheter end 104 into a bladder of a patient. Operation 208 of method 200 includes providing a flow from the fluid reservoir 102 to the bladder to begin the irrigation process in the nominal mode. As fluid flow is generated, a turbulent flow, a laminar flow, or a combination thereof is generated at the tip 126 of the catheter end 104.

Operation 212 of method 200 can include sensing the inflow fluid from the fluid reservoir. Sensing the fluid can be used to sense and adjust fluid flow to determine what type of inflow, outflow, and combination thereof is required at any given time to break down a clot or other organic material, evacuate a clot or other organic material, prevent new clot or organic material aggregation from forming, and do so in a manner that is relatively atraumatic to the surrounding tissue.

As described above, the flow can direct small particles of emulsified organic material to an evacuation lumen, such as the second channel 122 of the catheter, for drainage from the anatomic space. In particular, small particles of emulsified clot can be drained from the bladder into the waste fluid trap 108. Further, a shear force can be generated at the tip 126 of the catheter end 104, such as at operation 216 of method 200, to break down relatively large collections of organic material proximate to the catheter end 104 into smaller pieces that can more easily drain out of the bladder. In use, the irrigation system 100 can alternate between a clot breakdown flow and a clot flushing/draining flow. For example, at step 220 of method 200, fluid can be irrigated and drained from the bladder, while at operation 224 outflow fluid parameters are measured. The order of operations 204 to 224 are by way of example, and various operations can take place at the same (or substantially similar) time.

At decision 228 of method 200, an operator and/or sensors within the irrigation system 100 can determine if there is a clog present. If so, the system may be sent back to operation 208 to alternate between the nominal and maintenance modes to dislodge particles or organic material during the troubleshooting mode. If no remaining clogs or clots are present, the method 200 may terminate.

While embodiments of the invention may be used with a variety of suction devices or wall modules, other suction mechanisms are possible. In general, the use of a suction-irrigator to break down large clot volumes (e.g., greater than 100 mL) can require a substantial amount of fluid (e.g., over 5 liters). Large volumes of sterile saline, which may be used to fill a bladder to assist with clot evacuation, may be difficult to obtain. For example, some hospital settings may only provide 1 to 2 liters of sterile saline in vessel that may require a dedicated IV pole. Further, typical suction canisters for hospital wall suction devices, which can be used to collect aspirated clots and instilled saline, may range from 1.5 to 2 liters. As such, use of irrigation systems described herein may require more than one typical suction canister.

In this regard, in use, for example, a first canister may be swapped out for a new canister when the first canister is filled. Similarly, multiple vessels of saline may be required. This process may be repeated such that a plurality of canisters are filled during an irrigation process. In general, the process of swapping canisters can increase costs and time and may require a user to break sterility during the irrigation process. Alternatively, multiple canisters can be connected in series to increase the overall collection volume without requiring additional canisters to be added or swapped into the system during an irrigation process. In general, a series connection of canisters can require a large amount of space (e.g., in storage and in set-up) and may still require a significant amount of sterile saline throughout the procedure. Thus, in some embodiments, it may be useful to employ a fluid recycler.

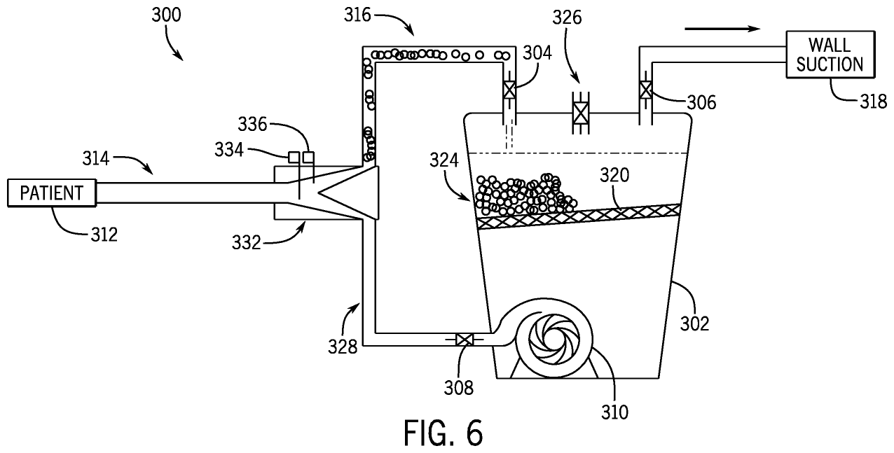

FIG. 6 illustrates an irrigation system 300 that includes a fluid recycler according to one embodiment of the invention. In some embodiments, the irrigation system 300 can include a suction canister 302 with three ports 304, 306, 308. Two of the three ports 304, 306 may be disposed at the top of the canister 302 and one port 308 may be disposed at the bottom of the canister 302 proximate to a base. The bottom port 308 may be in fluid communication with a pump or a fluid blower 310 that can pump fluid out of the canister 302 and into a patient 312 via a catheter 314 and tubing. The catheter 314 may be configured as the catheter described above with respect to FIGS. 1-4. One port 304 on the top can be connected to the patient 312 via tubing and the catheter 314 to provide an outflow pathway 316 during the irrigation process. For example, fluid, urine, clots, etc. may travel through the outflow pathway 316 from the patient 312 and into the canister 302. The other port 306 on the top of the canister 302 can be connected to a suction device 318 (e.g., a wall suction) to create a negative pressure vacuum in the canister 302. In some embodiments, an in-line regulator, sensor, and/or controller can be incorporated between the suction device 318 and the irrigation device 302 to regulate standard and safe levels of generated negative pressure.

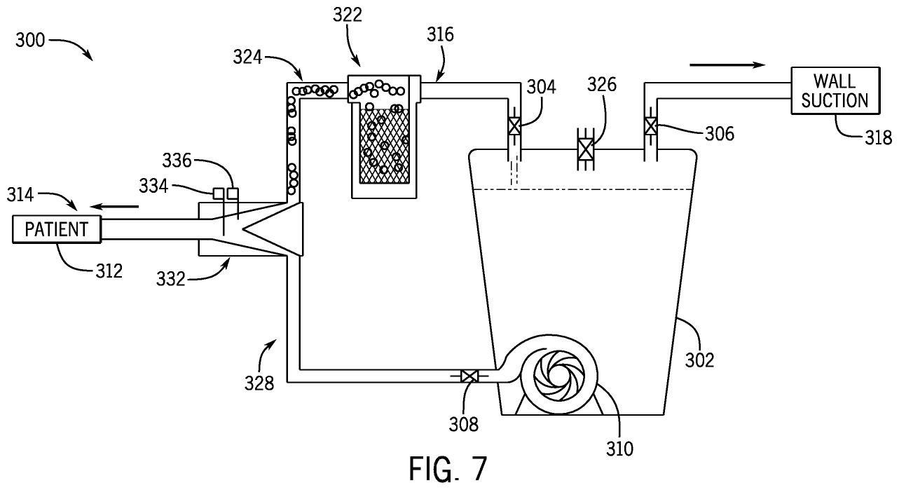

The irrigation system 300 can further include a filter 320. The filter 320 can allow fluid to pass from the inlet of the cannister on top (e.g., patient outflow via suction) to the outlet of the canister on the bottom (e.g., patient inflow). The filter 320 may be able to filter out clot particles and blood. Thus, the filter 320 allows fluid within the cannister to be reused while preventing an evacuated clot 324 from being re-instilled into the patient. Additionally, in some embodiments, the filter 320 may have a certain level of filtration that can clarify the recycled fluid which can allow better visual analysis of a remaining clot burden (e.g., based on how red the outflow from the patient is) and may filter out bacteria to help keep inflowing fluid sterile. Other filter configurations are possible, such as, for example, a T-filter 322 in line with the patient outflow suction tubing 316, illustrated in FIG. 7.

In general, the irrigation system 300 may be configured as a fluid recycler that pumps fluid from a vacuum chamber. In some embodiments, the irrigation system 300 can include a valve 326 on top of the fluid recycling cannister 302 that may allow venting of the canister 302. The valve 326 can allow the canister 302 to rapidly cycle between negative pressure for suction and atmospheric/gauge pressure for fluid instillation. The valve 326 may also be able to mitigate drastic pressure variation during use. In general, the valve 326 may be configured as an atmospheric decompression valve.

In some embodiments, the irrigation system 300 can be controlled via a handheld device 332 that can be immediately proximal to the catheter 314 used for clot evacuation. The handheld device 332 can include a plurality of independent channels, such as, for example, one for patient inflow, one for patient outflow via suction, and one for patient outflow via gravity. In some embodiments, the patient outflow channel via suction and the patient outflow via gravity may be configured as a single channel. The channels can coalesce to a common chamber leading to the catheter 314. A plurality of buttons (e.g., buttons 334, 336) on top of the handheld device 332 can be used to active patient outflow via suction, patient outflow via gravity, and saline inflow via a pump or blower. In some embodiments, a levered valve can be used to divert patient outflow to a standard urinary collection gravity bag versus into a depressurized outflow canister.

FIGS. 6-8 illustrate various embodiments of the irrigation system 300 that provide a fluid circuit to pump irrigant via the fluid pump 310 from the bottom of the cannister 302 through inflow tubing 328 into the patient 312 via the handheld device 332 and catheter 314 as controlled by an inflow valve controller button 334 of the handheld device 332. Fluid and clot can then be evacuated from the patient 312 via the catheter 314 and handheld device 332 as controlled by an outflow valve controller button 336 into the outflow tubing 316 back into the top of the canister 302. In FIG. 6, a clot 324 can then be filtered by a trans-canister filter 320 such that only irrigant settles to the bottom of the canister 302 to be reused by the fluid pump 310 for inflow. Pressure can be regulated by wall suction 318 to provide negative pressure for fluid outflow. The atmospheric decompression valve 326 can be used to depressurize the system when the fluid pump 310 is activated for fluid inflow.

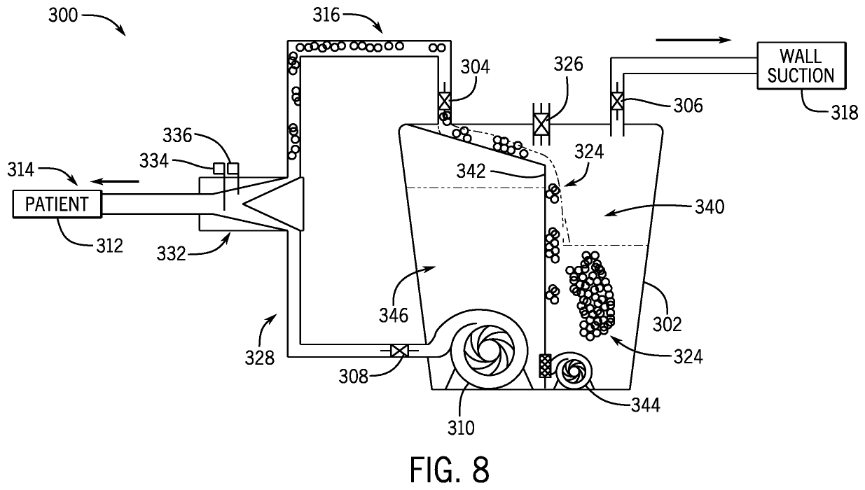

Illustrated in FIG. 7, fluid in the outflow tubing 316 can be filtered through the in-line T-filter 322 so that a clot is collected in the filter and filtered irrigant flows back into the top of the cannister. In another embodiment, FIG. 8 illustrates an outflow chamber 340. The outflow chamber 340 is separated within the canister 302 by a chamber separator 342. The chamber separator 342 can separate the interior of the canister 302 into the outflow chamber 340 and an inflow chamber 346. Fluid can be pumped through the outflow pathway 316 from the patient 312 and into the outflow chamber 340. A filter pump 344 can be used to pump outflow irrigant from the outflow chamber 340 into the inflow chamber 346. The filter pump 344 can allow irrigant to pass from the outflow chamber 340 into the inflow chamber 346 while filtering out clots and other debris so that irrigant in the inflow chamber 346 can be reused as inflow irrigant.

Within this specification embodiments have been described in a way which enables a clear and concise specification to be written, but it is intended and will be appreciated that embodiments may be variously combined or separated without parting from the invention. For example, it will be appreciated that all preferred features described herein are applicable to all aspects of the invention described herein.

Thus, while the invention has been described in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

1. An irrigation system comprising:
a tube manifold;
a fluid reservoir in fluid communication with the tube manifold;
a waste fluid trap in fluid communication with the tube manifold; and
an emulsification system arranged at a distal end portion of the tube manifold and configured to be inserted into an anatomic space and emulsify organic material located within the anatomic space.

2. The irrigation system of claim 1, wherein the fluid reservoir is in fluid communication with a flow module that is configured to provide a positive pressure in the tube manifold.

3. The irrigation system of claim 2, wherein the flow module generates a turbulent or laminar flow at the distal end portion of the tube manifold.

4. The irrigation system of claim 1, wherein the waste fluid trap is in fluid communication with a vacuum module that is configured to provide a negative pressure in the tube manifold.

5. The irrigation system of claim 1 further comprising:
a valve; and
a valve actuator configured to actuate the valve between fluidly coupling the reservoir and the distal end portion of the tube manifold and fluidly coupling the waste fluid trap and the distal end portion of the tube manifold.

6. The irrigation system of claim 5, wherein the valve actuator is configured to alternate positive and negative pressure flow in the tube manifold.

7. The irrigation system of claim 5, wherein the valve actuator controls negative pressure applied to the anatomic space.

8. The irrigation system of claim 5, wherein the valve actuator is in communication with a fluid sensor and is automatically actuated when the fluid sensor signal crosses a predetermined threshold.

9. The irrigation system of claim 8, wherein the fluid sensor senses one of a volumetric flow rate, a mass flow rate, or a pressure differential.

10. The irrigation system of claim 1, wherein the emulsification system includes a network of bars and comprises material including at least one of nylon, metal, or natural fibers.

11. The irrigation system of claim 1, wherein the emulsification system includes at least one of:
a blade element disposed within the tube manifold,
a nozzle in fluid communication with the fluid reservoir;
an agitator that is configured to generate a mechanical force against organic material in the anatomic space.

12. (canceled)

13. (canceled)

14. The irrigation system of claim 11, wherein the emulsification system includes a cage configured to surround the agitator to allow organic material in the anatomic space to reach the agitator and prevent the agitator from engaging vital tissue forming the anatomic space.

15. The irrigation system of claim 1, wherein the tube manifold forms a single-lumen catheter at a distal end.

16. The irrigation system of claim 1, wherein the tube manifold includes a multi-lumen catheter having a first channel and a second channel,
wherein the first channel is in alternating fluid communication with the fluid reservoir and the waste fluid trap, and
wherein the second channel is dimensioned to receive a balloon.

17. The irrigation system of claim 16, wherein the balloon is inflated with a fluid to dynamically measure pressure within the anatomic space.

18. The irrigation system of claim 16, wherein the multi-lumen catheter includes a third channel in continuous fluid communication with a fluid source.

19. The irrigation system of claim 1, wherein a distal end of the tube manifold forms a multi-lumen catheter including a first channel in fluid communication with the fluid reservoir, a second channel in fluid communication with the waste fluid trap, and a third channel providing access to a balloon.

20. A method of hematuria irrigation, the method comprising:
inserting a catheter into a bladder of a patient;
introducing an irrigant into the bladder through the catheter;
causing solid or semi-solid organic material to engage an emulsification system; and
draining the irrigant and organic material from the bladder into a waste fluid trap via a vacuum module.

21. The method of claim 20, further comprising propelling the irrigant to create a flow that is configured to emulsify organic material within the bladder.

22. The method of claim 20, further comprising simultaneously or sequentially introducing the irrigant into the bladder and draining the irrigant and the organic material.

23. The method of claim 20, further comprising controlling an actuator connected to the catheter to introduce the irrigant into bladder and drain the irrigant from the bladder through a common channel in the catheter.

24. The method of claim 20, further comprising introducing the irrigant into the bladder through a first channel and draining the irrigant from the bladder through a second channel.

25. The method of claim 20, further comprising introducing an agitator into the bladder after the irrigant is propelled into the bladder.

26. (canceled)

27. An irrigation system having a fluid recycler system, comprising:
a pump;
a pressure regulator;
a fluid reservoir configured to receive patient aspirate and irrigant;
a filter in fluid communication with the fluid reservoir to separate the patient aspirate from the irrigant;
a port fluidly coupled to a suction generator; and
a pressure sensor configured to monitor pressure of the recycler system.

28. A handheld device for use with a fluid recycler system, the handheld device comprising:
a first channel fluidly coupled to a fluid reservoir of the recycler system in an inflow passageway;
a second channel fluidly coupled to the fluid reservoir in an outflow passageway;
a fluid chamber in fluid communication with each of the first channel, the second channel, and a catheter; and
a control switch configured to selectively allow:
fluid flow through the inflow passageway via the first channel; or
fluid flow through the outflow passageway via the second channel.
